(19) United States
(12) Patent Application Publication
(10) Pub. No.: US 2004/0137642 A1
(43) Pub. Date: Jul. 15, 2004

(54) MHC tetramers

(76) Inventors: Volker Erfle, Munchen (DE); Horst Wolff, Munchen (DE); Dirk Busch, Munchen (DE); Markus Neumann, Munchen (DE)

Correspondence Address: JENKINS & WILSON, PA, 3100 TOWER BLVD, SUITE 1400, DURHAM, NC, 27707, US

(21) Appl. No.: 10/682,675

(22) Filed: Oct. 9, 2003

(30) Foreign Application Priority Data
Apr. 10, 2002 (WO) PCT/EP02/03995
Apr. 10, 2001 (DE) 10117858.1

(51) Int. Cl. C07K 14/74; G01N 33/543

(52) U.S. Cl. 436/518; 530/350

(57) ABSTRACT

The invention relates to MHC tetramer fusion proteins, MHC monomer fusion proteins, DNA encoding a MHC fusion protein as well as RNA yielding after transcription a MHC monomer fusion protein. Furthermore, the invention discloses the use of a DsRed protein as an agent for the tetramerization of MHC molecules, methods for the preparation of fusion proteins containing MHC and DsRed, and methods for the preparation of MHC tetramers. Moreover, the invention describes methods for the examination of an antigen-specific cellular immune response, particularly for the detection of T lymphocytes carrying specific T cell receptors on their surfaces, the uses of the MHC monomers and tetramers prepared according to the invention as well as test systems containing the MHC monomers or MHC tetramers according to the invention.

### RELATED APPLICATIONS

This application is a continuation of PCT patent application number PCT/EP02/03995, filed Apr. 10, 2002, which claims priority to German patent application number 101 17 858.1, filed Apr. 10, 2001, the disclosures of each of which are incorporated herein by reference in their entirety.

### TECHNICAL FIELD

The invention relates to MHC tetramer fusion proteins, MHC monomer fusion proteins, DNA encoding a MHC fusion protein as well as RNA which after transcription yields a MHC monomer fusion protein. Furthermore, the invention discloses the use of a DsRed protein as an agent for the tetramerization of MHC molecules, processes for the preparation of fusion proteins containing MHC and DsRed, and processes for the preparation of MHC tetramers. Moreover, the invention describes methods for the analysis of an antigen-specific cellular immune response, particularly for the detection of T lymphocytes carrying specific T cell receptors on their cell surface, the use of the MHC monomers and tetramers prepared according to the invention as well as test systems containing the MHC monomers or MHC tetramers according to the invention.

### BACKGROUND ART

The recognition of antigenic structures by the cellular immune system is mediated by surface molecules of the “major histocompatibility complex” (MHC). Antigen-presenting cells (APCs) process antigens into short peptides which after binding are presented in a specific peptide binding fold of the MHC molecule and thus can be recognized by the T cells. Specific recognition of the epitope (peptide fragment) by the T cell receptor (TCR) requires simultaneous interaction with the MHC molecule (“MHC restriction”).

The binding of MHC/peptide complexes to the TCR is characterized by a very low affinity, particularly due to a very fast dissociation (K^{off}) of the MHC from the TCR. For this reason it is impossible to label T cells directly by means of a soluble form of the natural ligand (e.g. in the form of a fluorescence-labeled MHC/peptide complex) in accordance with their epitope specificity. By multimerization of MHC/peptide complexes, for example into MHC tetramers, it could be demonstrated that the relative avidity of the epitope-specific binding at the T cell surface can be increased to an extent to enable a specific T cell labeling. For this purpose, soluble MHC molecules are generated in vitro, specifically biotinylated and multimerized via fluorescence-labeled streptavidin. Using such reagents, the antigen-specific cellular immune response may be analyzed in great detail in the animal model or directly in man.

The preparation of MHC tetramer reagents involves a number of complex biochemical reactions wherein proteins expressed in a recombinant manner generally must be folded correctly in vitro, biotinylated and afterwards caused to form tetramers in the correct molar ratio.

At present, most MHC tetramer reagents are prepared using a very complex and difficult process. First, MHC components are expressed in the form of recombinant proteins in E. coli and purified from inclusion bodies.

Following urea denaturation, the MHC portions are folded in the presence of high peptide/epitope concentrations by dilution in an arginine-rich buffer which contains a glutathione redox system, and are subsequently isolated. In a further step, the recombinant MHC is biotinylated and, after a second purification, is multimerized via streptavidin. The streptavidin used for multimerization is labeled with phycoerythrine for later optical detection. These fluorescence-coupled multimeric MHC reagents may be incubated with complex T cell mixtures, and in this manner the MHC/peptide-specific cells within the total population can be determined (e.g. by FACS analysis).

The present technique for the preparation of MHC multimer reagents is very difficult, vulnerable (e.g. with respect to the efficiency of the biotinylation reaction) and costly. A simplification of the process of preparation would significantly promote the broader use of this methodology in basic research and also in the clinical diagnostic field.

### SUMMARY OF THE INVENTION

Therefore, it is an object of the present invention to provide a novel agent which improves the tetramerization of MHC molecules and avoids the disadvantages described above.

This object has been achieved according to the invention by the MHC tetramerization agent described in more detail in claim 1. Preferred embodiments of the invention become apparent from the dependent claims as well as from the further independent claims.

In the following, the present invention will be explained in more detail with respect to individual embodiments. However, the invention is not limited to these specific embodiments but the scope of the invention is defined by the claims in connection with the specification.

According to the invention tetramer formation is carried out starting with MHC molecules and using the DsRed protein. DsRed is a protein emitting a red fluorescence which is obtained from the sea anemone Discosoma sp., and as the Green Fluorescent Protein (GFP) it belongs to a family of fluorescent proteins. Merely as an example, reference is made herein to the publication of Matz, M. V., et al., Nature Biotech. 17: 969-973, 1999. The structure of DsRed is known, and it can obtained in a recombinant form for example from CLONTECH^{R}. It is further known that DsRed can form tetramers in vivo as well as in vitro. The structure of these tetramers is also known (Nature Structural Biology, 2000, pages 1133-1138).

Now, it has been discovered surprisingly and unexpectedly by the present inventors that DsRed may be used also for the tetramerization of MHC molecules and at the same time for the subsequent optical detection of the agent. Neither binding of the antigen-specific peptide to the MHC molecules nor binding of the MHC tetramers to the T cell receptors of a T cell is disturbed or adversely affected by this DsRed-mediated tetramerization. Particularly, by the DsRed-mediated tetramerization the preparation of tetramers can essentially be simplified, accelerated and made more efficient in a cost-effective manner without hampering the functionality. The detection of the DsRed protein is carried out by fluorescence detection methods in a manner known per se, as for example those described in the prior art such as in the protocols published by CLONTECH^{R} laboratories, Inc. (CLONTECHniques XIV (4): 2-6). These include not only FACS analyses but also fluorescence microscopy and fluorescence-based scanning procedures (e.g. Fluorimanager by Molecular Dynamics).

According to the invention the fluorescent DsRed protein is used for the tetramerization of both MHC class I and MHC class II molecules.

Examples of useful histocompatibility antigens are MHC class I antigens, for example HLA-A (such as A1, A2, A3, All, A24, A31, A33, and A38), HLA-B and HLA-C, MHC class II antigens such as HLA-DR, HLA-DQ, HLA-DX, HLA-DO, HLA-DZ, and HLA-DP.

MHC tetramers are complexes consisting of four MHC molecules which may associated with a specific peptide and can be detected due to their binding to a fluorochrome. These complexes bind to a specific group of T cell receptors on CD8⁺ T cells. If the tetramers thus obtained are mixed with PBLs or whole blood and are detected using flow cytometrical methods, the amount of all T cells specific for one peptide and the corresponding allele may be analyzed. Thus, it is possible to determine the cellular immunity against a specific peptide.

MHC tetramers according to the invention may be for example used to examine the cellular immunity under the following conditions:

1. in the case of all viral infections for example HIV, HBV, CMV, HPV, HBV, HCV, influenza and measles, and many others;

2. bacterial infections;

3. parasitic infections such as malaria;

4. tumors including breast, prostate, melanoma, colon, lung and cervix tumors;

5. autoimmunity diseases including multiple sclerosis, diabetes, rheumatoid arthritis etc.;

6. allergic diseases such as asthma bronchiale, neurodermitis etc.

By means of the tetramers technology it will be possible to identify individual T cells on the basis of their binding specificity to the MHC/peptide complex. Due to the specificity of the tetramers the following advantages will be obtained:

the method is quantitative;

no radioactive labels have to be used;

the method is fast so that also fresh blood samples or tissue culture samples can be analyzed, and large numbers of samples can be assessed;

by the use of a flow cytometer the cells can be labeled at the same time not only with the tetrameric DsRed fluorochrome but also with other cell surface markers;

uniform subpopulations can be sorted by means of flow cytometry and examined for their functionalities by means of other test systems;

specific T cells from blood samples can be analyzed without previous in vitro culturing;

all specific T cells can be detected disregarding their functional status, for example cytotoxic T cells, T helper cells etc.

The invention is based on the preparation of a fusion protein consisting of a gene encoding a MHC protein and a gene encoding a DsRed protein. For this purpose, in the case of the MHC molecule the transmembrane and cytosolic portions are preferably removed to obtain a soluble form of the MHC molecule. The MHC portion and the DsRed portion are preferably bound to each other by means of a linker molecule. The truncated MHC molecule obtained after removal of the transmembrane and cytosolic portions is then coupled to the N terminus of DsRed via the linker molecule.

It has to be specifically pointed out that the formation of tetramers via the DsRed portion is not hampered by the introduction of the linker molecule. Since the spatial structure of the DsRed molecule is known (two N terminal regions each are facing each other) the linker molecules can be designed appropriately. It is particularly preferred to use an amino acid linker. An example of a flexible amino acid linker is a derivative of the lacZ-alpha peptide [as wrote in the “single letter code”: MASSG GTGGS GGTGG SGGGG ASPSL VPSSD PLVTA ASVLE FALAG AQE], or the synthetic flexible linker Gly Gly Gly Ser Gly Gly Gly Thr[Gly Gly Ser Gly Gly Thr] 3 which is introduced between the two protein portions of the MHC/DsRed fusion protein and does not hamper tetramerization sterically.

It should be understood, however, that also other amino acid linkers are available to those skilled in the art.

The corresponding techniques are well known. It is also possible, however, to connect the proteins themselves for example by peptide chemical bonds using cross linkers or similar techniques. Reference is made for example to Sambrook et al., Molecular Cloning: A Laboratory Manual (Cold Spring Harbor Laboratory, Cold Spring Harbor, N.Y.), 1989 and Ansubel F. M. et al., 1994, Current Protocols in Molecular Biology (John Wiley and Sons, NY) and the later editions.

In a preferred embodiment of the invention also a variation of the amino acid linker may be used which contains at least a recognition sequence for a protease, for example factor Xa. This enables a simplified cleavage of the MHC molecules from the DsRed tetramers.

The following further variations of the linker between MHC and DsRed can be used in the context of the present invention:

lacZ linker:

E Q A G A L A F E L V S A A T V L P D S S P V L S

standard serine-glycine linker:

S G G S S G G G

extremely long standard linker:

S S S G G G S S G G S S G G G

The recombinant MHC/DsRed DNA molecules prepared according to the invention can be cloned in a manner known per se in a recombinant form into expression plasmids and expressed in prokaryotic cells, preferably in E. coli cells, or in eukaryotic cells, for example in yeast cells or in established human cells. Suitable techniques are available in the field, and reference is made again to the laboratory manual cited above. The recombinant monomeric DsRed/MHC protein molecules obtained in this manner are purified by methods known per se and may be tetramerized in vitro or in vivo.

To carry out the tetramerization, the soluble versions of the MHC/DsRed molecules, optionally in combination with β₂ microglobulin particularly in the case of MHC I, are caused to tetramerize. The tetramerization preferably takes place in the presence of the antigen-specific peptide. The formation of tetramers is an intrinsic property of the DsRed protein, and therefore none of the complex and time-consuming methods known from the prior art such as biotinylations, streptavidin linkages as well as fluorochrome coupling reactions, have to be used any more for ensuring tetramers formation and fluorescence development. In the present invention the DsRed protein employed serves both as a tetramerizing agent and as a fluorescent agent.

It has to be understood, however, that the DsRed protein known per se may be modified by methods known per se for example to improve the tetramerization, binding to the MHC molecule and/or the fluorescence activity. Generally, to carry out such mutagenizations randomly generated mutants are tested for their new characteristics, or alternatively after a structural analysis a mutagenization of the DsRed protein is carried out in a site-specific manner to improve its activities. It has to be understood, however, that also other proteins similar to DsRed may be used which have been obtained from other organisms. This requires, however, that they are capable of forming tetramers or multimers, respectively, and have fluorescence activity.

Thus, for example several different mutations may be introduced into the DsRed protein to enhance its usefulness within the fusion protein or to create completely new possibilities of use, respectively.

Several of these DsRed mutants have already been tested by the present inventors in different fields of use, others have been published by other work groups in a different context, and still others are based on theoretical considerations on the basis of the spatial structure known per se and the knowledge of the biochemistry of fluorescent proteins.

Basically, one considers mainly those mutations which accelerate or enhance the generation of fluorescence as well as those improving the specific tetramerization properties and reducing the unspecific aggregation properties. Furthermore, those mutants are of interest which alter the spectral properties since this may provide novel possibilities of use in the detection of MHC tetramers. Particularly, shifts of the emission further into the region of longer wavelengths might be advantageous since this could simplify fluorescence detection by means of FACS.

All amino acids are given in the standard single letter code:

Improvement/Alteration of the Tetramerization Properties

R2A, K5E, K9T: inhibition of unspecific protein aggregates;

addition of several hydrophobic amino acids directly at the C terminus of the protein to stabilize the tetramerization such as the octapeptide “L L I L A I L H”;

exchange of problematic amino acids not required for the protein structure by more suitable aas such as the replacement of R and K by S or of F by T and the like.

Alteration of the Fluorescence Properties

A105V, 1161T, S197A: improvement of chromophore formation;

V105A, S197T: color changing mutant;

K83M, K83R, Y 120H, K83W, alteration of the emission K70R, Y38L, H41W, N42D or excitation properties, respectively

The abbreviations mentioned above mean that e.g. R2A represents a replacement of the amino acid R in position 2 in the total sequence by the amino acid A.

In summary, the terms “DsRed fluorescent protein” or “recombinant DsRed fluorescent protein” are not limited to a specific protein but comprise all variations and derivatives of the known DsRed sequence which are capable of performing its functions in the context of the present invention, i.e. which are suitable for the tetramerization of MHC molecules as well as for optical detection. Preferred examples are mentioned above while it should be understood that these examples shall not be considered only by themselves but that at any time the terms “DsRed fluorescent protein” or “recombinant DsRed fluorescent protein” also comprise combinations of the individual changes in order to achieve an advantageous DsRed protein.

Modifications of the sequences such as for example deletions, insertions, or substitutions within the sequence generating so-called “silent” changes in the protein molecules thus obtained are also considered as falling within the scope of the present invention.

Preferably, such amino acid substitutions are the result of an replacement of one amino acid by another amino acid having similar structural and/or chemical properties, i.e. a conservative amino acid substitution. Amino acid substitutions may be carried out on the basis of similarities in polarity, charge, solubility, hydrophobicity, hydrophilicity and/or the amphipathic (amphiphilic) nature of the residues involved. Examples of apolar (hydrophobic) amino acids are alanine, leucine, isoleucine, valine, proline, phenylalanine, tryptophane, and methionine. Polar neutral amino acids include glycine, serine, threonine, cysteine, tyrosine, asparagine, and glutamine. Positively charged (basic) amino acids include arginine, lysine, and histidine. And negatively charged (acidic) amino acids include aspartic acid and glutamic acid.

“Insertions” or “deletions” typically are in the range of one to five amino acids. The degree of variation allowed may be determined experimentally by systematically performed insertions, deletions, or substitutions of amino acids within a polypeptide molecule using DNA engineering techniques and by examining the recombinant variations obtained with respect to their biological activity.

This means that where the term “DsRed protein” is used either in the specification or in the claims it comprises all those modifications and variations which result in a biologically equivalent DsRed protein.

The tetramers formed according to the invention are mixed with the cellular population to be analyzed. By cell population there are particularly meant populations of PBMCs (peripheral blood mononuclear cells)=or T lymphocytes (T cells). Only those T cells having T cell receptors capable of binding to the specific MHC/peptide combination present in the tetramer are able to bind to the tetramer. Such cells are labeled by the DsRed fluorochrome. It should be understood, however, that also other fluorescent labels in addition to DsRed may be employed. For example a monoclonal antibody specific for a T cell marker may be used in combination with a different fluorochrome, such as FITC. In this case, the cells may be analyzed using a flow cytometrical method. The portion of the CD8⁺ T cell population which has been positively stained using the tetramers is determined. Also, by means of fluorescence detection methods, for example flow cytometry, the MHC/tetramer/T cell complexes can be sorted, and the T cells obtained in this manner may be for example applied to a patient.

In another embodiment of the invention the MHC monomers or tetramers are present in a test system which may be presented in the form of a kit.

The approach of the solution according to the invention reduces the components required for the preparation of MHC multimer reagents (2 MHC chains [e.g. MHC I: heavy chain and beta 2 microglobulin, MHC II: alpha and beta chain], d-biotin, peptide, streptavidin-PE) to 4 (MHC/linker/DsRed fusion protein, 2^{nd} MHC chain and the respective MHC-binding peptide/epitope). Furthermore, the high number of reaction sequences required (extraction of recombinant MHC portions, in vitro folding in the presence of peptide, biotinylation, chromatographic separation, multimerization by addition of streptavidin-PE, further purification by molecular sieve chromatography) is reduced to e.g. 3 (extraction of recombinant HLA/linker/DsRed, refolding in the presence of peptide, and subsequent purification). The reduction alone of the number of purifications steps which are always accompanied by severe losses increases the total yield of reagent markedly. Furthermore, the MHC multimer complex generated is relatively small and much more stable which is advantageous over the reagents used up to now.

For the above-mentioned reasons the following possibilities are provided for the in vivo application of DsRed/MHC tetramers:

As described above, conventional MHC tetramers reagents are generally prepared using avidin or streptavidin. However, avidin or streptavidin, respectively, has a relatively short half life in vivo following intravenous application particularly due to its fast absorption in the liver. Accordingly, also conventional MHC tetramers reagents have only a rather short half life in vivo. For DsRed/MHC tetramers a significantly longer in vivo half life is expected; in addition, the fluorescent dye itself is very resistant to damaging or degrading effects in vivo. In vivo applications of MHC multimers are of importance both for the problems of basic research (e.g. in vivo staining of antigen-specific T cells and subsequent detection in a tissue section, antigen-specific immunization or tolerance induction, respectively) and for clinical applications (antigen-specific immunization or tolerance induction, respectively, conjugation of reagents with immune modulatory substances or tracers, respectively).

### BRIEF DESCRIPTION OF THE DRAWINGS

In the following, the present invention will be described with respect to Examples and the accompanying drawings which show:

FIG. 1: A schematic representation of a MHC-DsRed expression cassette in vector pet3a. This construct is presented as an example of several other variations. The individual components are represented in different colors. Blue: promoter or terminator, respectively, for the overexpression in E. coli; red: reading frame of a mutant DsRed protein; grey: reading frame of the MHC protein heavy chain; green: fusion protein of MHC and DsRed; cyan: linker region, in this case represented by a peptide which at the same time serves as an epitope tag. It has to be noted, however, that the reading frame of DsRed lacks an internal start codon whereby practically only the fusion protein in its full length can be expressed.

FIG. 2: A schematic representation of the essential components of another MHC-DsRed expression cassette. In contrast to the construct of FIG. 1 in this construct the linker peptide does not serve as an epitope tag but the construct has a linker composed of a number of serines and glycines. In this case, the flexibility of the linker and the mobility of the proteins connected to each other probably is significantly higher.

FIG. 3: MHC protein (heavy chain and β₂ microglobulin).

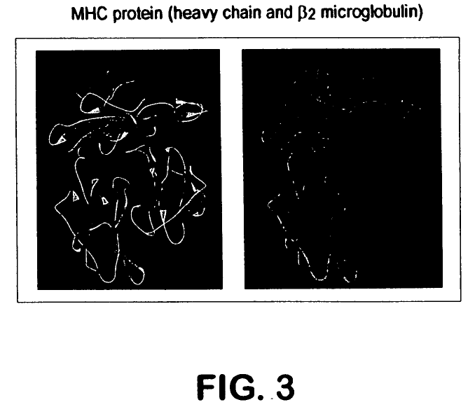

To the left is shown a representation of the secondary structure of the protein which is also that used in FIGS. 5 and 6 depicting the fusion proteins (helical structures are drawn in red, β sheets in blue, unstructured regions and turns in white). To the right the heavy chain (in green) and β₂ microgloblin (in purple) have been depicted separately for clarification.

FIG. 4: Spatial illustration of the DsRed1 tetramer. The individual chains of the homotetramer are drawn in the colors orange, red, green and cyan. A side view of the tetramer is shown to the right, a top view to the left. It can be clearly seen that the tetramer has the symmetrical and compact arrangement which renders it suitable for the intended application.

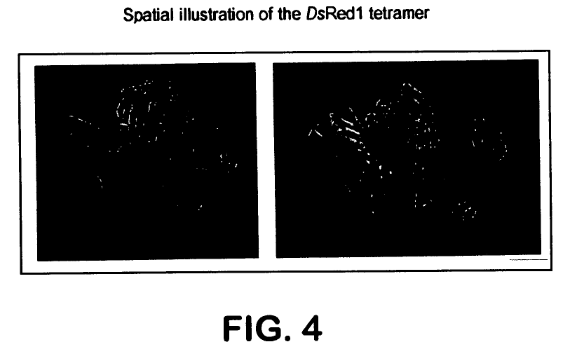

FIG. 5: MHC/DsRed tetramer. The MHC tetramer combination was prepared according to the descriptions of FIGS. 4 and 5. The C terminus of one MHC protein each was joined to the N terminus of one subunit of the DsRed tetramer in a computer-aided fashion.

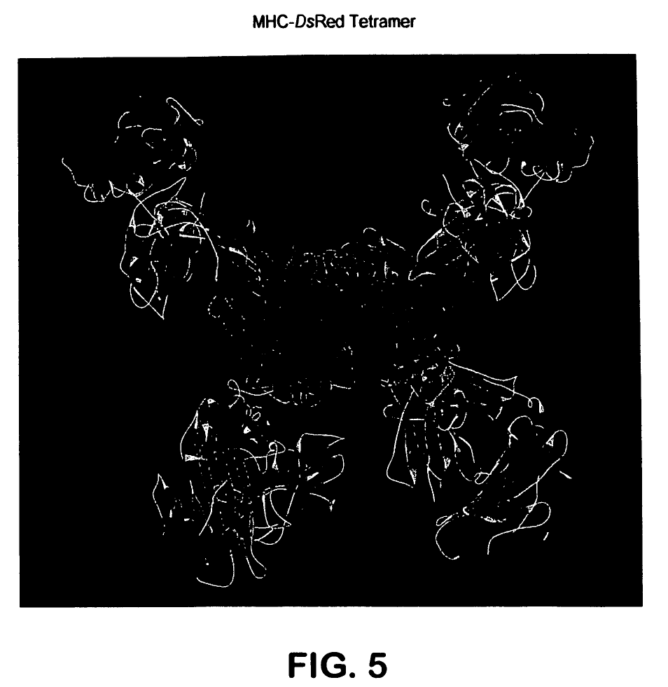

The representation only shows one possible spatial arrangement of the MHC molecules which are provided with flexibility of movement due to the flexible peptide linker. A side view of the DsRed tetramer is shown.

FIG. 6: MHC/DsRed tetramer. For explanations see FIG. 5. This view shows the DsRed tetramer from the top.

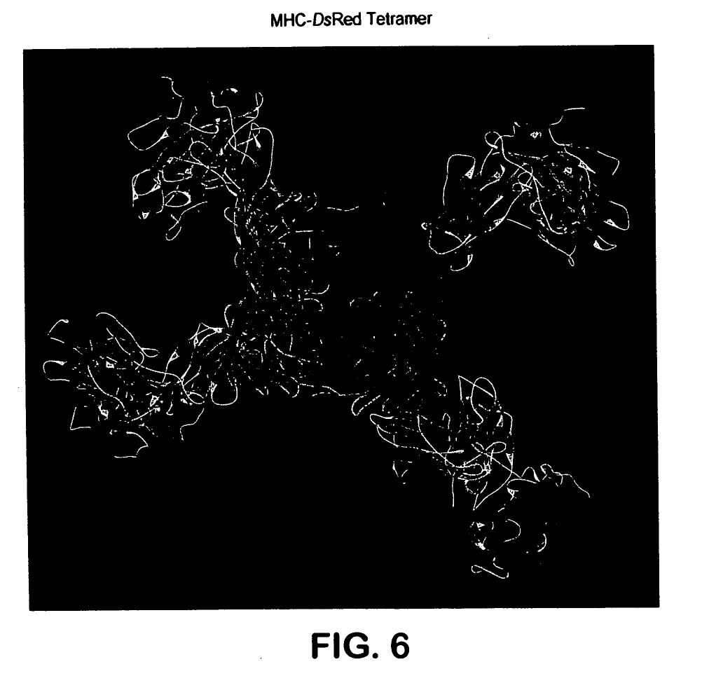

FIG. 7: Bacterial colonies expressing MHC/DsRed fusion protein. BL21 (DE3) bacteria were transformed with pET3a/H2-K^{d}-DsRed and cultured on agarose containing ampicillin. Individual bacterial colonies can be recognized by exhibiting a deep red color. The red color is due to the DsRed portion of the fusion protein which is expressed in a recombinant manner.

FIG. 8: Recombinant expression of the MHC/DsRed fusion protein. BL21 (DE3) bacteria were transformed with pET3a/H2-K^{d}-DsRed. After growth in liquid culture (LB, 100 μg/ml carbenicillin) an aliquot was removed (“blank”) at an OD₆₀₀ of about 0.8, and the remainder was incubated for further 3 hours in the presence of IPTG (0,4 mM). Samples of the “blank” and of “3 h” were subsequently washed, lysed in dH₂O, boiled in protein gel sample buffer and afterwards separated using SDS PAGE. The Figure shows a protein gel after Coomassie staining of two different cultures (left: MHC fusion protein with wild-type DsRed and right: with a DsRed mutant to optimize the folding properties). It should be noted that a clear additional band after 3 h of IPTG induction at about 65 kD corresponds to the recombinant fusion protein.

### EXAMPLES

Due to the superior fluorescence and tetramerization characteristics in various tests, experiments were carried out to achieve the formation of MHC tetramers by fusion to the red fluorescent protein DsRed.

Cloning of the Expression Constructs:

All expression constructs were generated by standard cloning techniques starting from vector pDSRed1-N1 (Clontech) which served as a PCR template for all other variations of the DsRed protein.

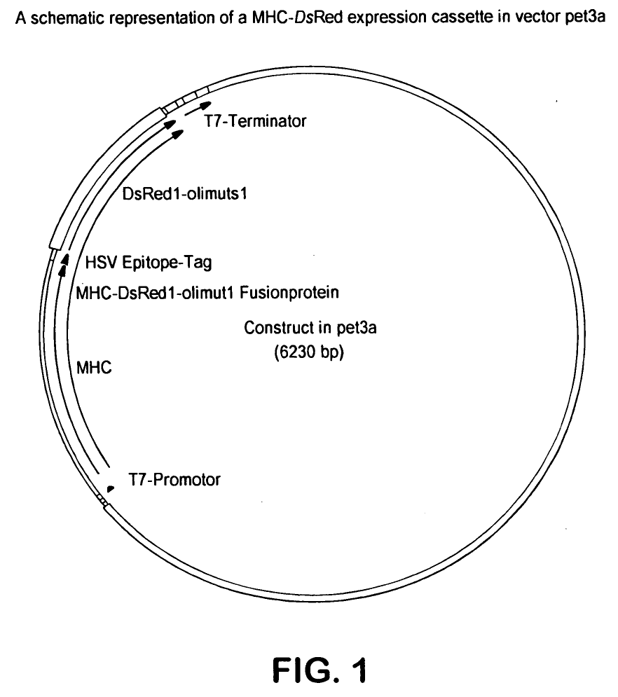

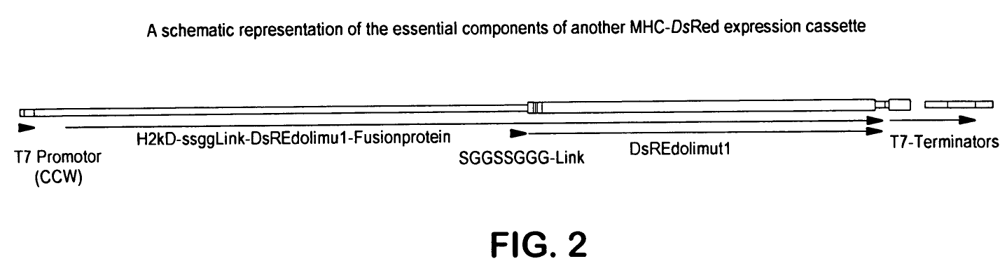

As an expression vector for the complete product and as a source of the MHC protein vector pET3a/H2-K^{d} was used. By means of various cloning strategies utilizing restriction sites already present in the target vector pET3a/H2-K^{d}, the DNA encoding various variations of DsRed was introduced. In this manner vectors were generated (such as pET3a/H2-K^{d}-DsRed) which contained a fusion protein of MHC and a DsRed variation under the control of a T7 promoter coupled to each other via different linker peptides (see FIGS. 1 and 2).

Only the starting ATG of the MHC protein was present as a translational start while no internal start codon at the linker or DsRed was present. The expression of truncated proteins can be largely excluded in this way.

The following sequence out of vector pET3a/H2-K^{d}-SSGGlinkDsRed exemplarily shows the sequence of the amino acids of the last 12 aas of the MHC molecule and the first 12 aas of a DsRed variation (in italics). In between there is a linker peptide used in this case (printed in bold).

L P P S T V S N T A S G G S S G G G V A S S E N V I T E F M

The next sequence derived from pET3a/H2-K^{d}-directDsRed again shows the sequence of the amino acids of the last 12 aas of the MHC molecule and of the first 12 aas of a DsRed variation (in italics). In between there is an epitope tag which can function as a linker and which furthermore can be detected due to its ability to bind to specific antibodies (printed in bold).

K L P P S T V S N T A S Q P E L A P E D P E D G G H D K V R S S K N V I K E F M

For a detailed description of the MHC/DsRed expression cassette see FIGS. 1 and 2.

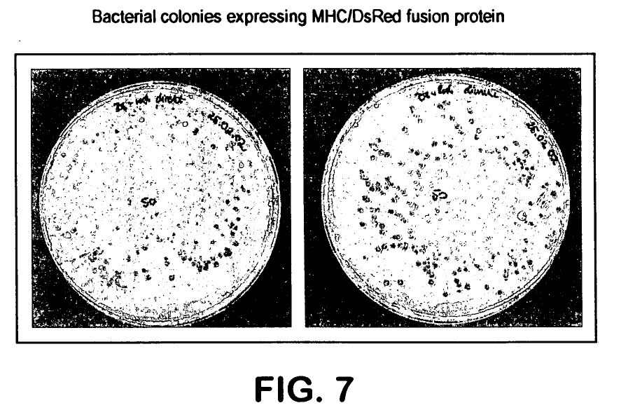

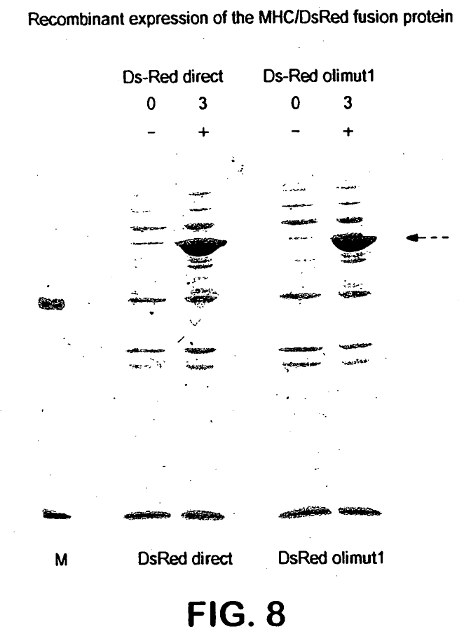

DsRed/MHC fusion proteins were cloned in pET3a (Novagen) vectors and afterwards transformed into BL21 (DE3) bacteria for expression. However, in this system a slight generation of a recombinant protein can be observed also without a specific induction of the expression. This basal expression of the DsRed fusion protein can be observed just directly in the bacterial colonies which after a short period show a deep red color (FIG. 7). If the transcription of the recombinant protein is induced by the addition of IPTG an overexpression of the recombinant protein takes place (FIG. 8). The preparation of large amounts of recombinant MHC/DsRed fusion protein could be achieved both using the original sequence of DsRed and using DsRed mutants (FIG. 8). Generally, to prepare recombinant MHC class I molecules the partial components (heavy chain and β₂ microglobulin) expressed in and purified from “inclusion bodies” are refolded in vitro in the presence of high concentrations of the respective MHC-binding peptide (epitope). If DsRed is expressed in the same way in “inclusion bodies” and purified and refolded in the same manner in vitro, a coloring protein is obtained which has all the characteristics of the properly folded fluorescent DsRed protein.

In summary these data show that two essential preconditions for a successful use of DsRed MHC tetramers have been fulfilled:

(1) Large amounts of MHC/DsRed fusion protein can be prepared in the bacterial expression system; in this case the in vivo expression already reveals the “functionality” of the DsRed portion of the fusion protein by the typical coloring characteristic;

(2) fluorescent DsRed protein may be generated from “inclusion bodies” under the same conditions as those which have been optimized for the preparation of soluble MHC molecules.

What is claimed is:

1. A MHC tetramer containing four MHC molecules as well as a tetramerization agent wherein it is formed from four MHC monomer fusion proteins, in which the fluorescent DsRed protein is coupled to the MHC protein via a linker molecule.

2. MHC tetramer according to claim 1 wherein said linker molecule consists of several amino acids.

3. A MHC tetramer according to claim 2 wherein said linker molecule is designed to enable a cleavage of the MHC molecules from the DsRed protein.

4. A MHC tetramer according to claim 3 wherein said linker molecule contains a protease recognition sequence.

5. A MHC tetramer according to claim 1 wherein the N terminal portions of the DsRed protein are facing each other during tetramer formation.

6. A MHC tetramer according to claim 1 wherein said MHC molecule is truncated.

7. A MHC tetramer according to claim 1 wherein said MHC molecule is truncated by a deletion of the transmembrane and/or the cytosolic portion.

8. A MHC tetramer according to claim 1 wherein it is present in the form of an epitope-specific DsRed-MHC-peptide complex, optionally in association with a T cell receptor.

9. A MHC tetramer according to claim 1 wherein said MHC molecule is a mammalian, preferably murine or human MHC molecule.

10. A MHC monomer fusion protein wherein it is present in association with a fluorescent DsRed protein, optionally linked via a linker molecule.

11. A fusion protein according to claim 10 wherein said MHC molecule is truncated.

12. A fusion protein according to claim 11 wherein said MHC molecule is truncated by a deletion of the transmembrane and/or the cytosolic portion.

13. A DNA encoding a fusion protein according to claim 10.

14. An RNA yielding after transcription a fusion protein according to claim 10.

15. A prokaryotic or eukaryotic cell containing a DNA or an RNA according to claim 1.

16. The use of a DsRed protein as an agent for the tetramerization or multimerization of MHC molecules.

17. A method for the preparation of a fusion protein according to claim 10 wherein:
a) a DNA encoding a MHC protein is linked, optionally via a linker, to a DNA encoding a DsRed protein to yield a DNA encoding a fusion protein and the fusion protein is expressed; or
a MHC protein is linked, optionally via a linker, to a DsRed protein to yield a fusion protein;
b) optionally the fusion protein is purified.

18. A method for the preparation of MHC tetramers wherein:
a) a DNA encoding a MHC protein is linked, optionally via a linker, to a DNA encoding a DsRed protein to yield a DNA encoding a fusion protein and the fusion protein is expressed; or
a MHC protein is linked, optionally via a linker, to a DsRed protein to yield a fusion protein;
b) optionally the fusion protein is purified;
c) the MHC fusion protein is tetramerized, optionally in the presence of the antigen-specific peptides.

19. A method according to claim 18 wherein the tetramerization is carried out in the presence of 2 microglobulin.

20. A method for the examination of an antigen-specific cellular immune response wherein:
a) a MHC tetramer or its monomeric form according to claim 1 is contacted with T cells, preferably with CD⁸⁺ T cells, in the presence of a peptide to obtain T-antigen-MHC tetramers;
b) the tetramers obtained are detected, preferably by means of a flow-cytometric method or other detection procedures suitable for the detection of fluorescence emissions, such as fluorescence microscopy or fluorescence scanning procedures.

21. A method according to claim 20 wherein:
a) the T cells are used as present in whole blood or PBLs; or
b) the T cells obtained from lymph nodes or extralymphoid tissues, respectively, are used.

22. A test system containing MHC monomers or MHC tetramers according to claim 1.

23. The use of MHC tetramers according to claim 1 for the examination of an antigen-specific immune response, the detection or sorting of T cells carrying specific T cell receptors on their surfaces, and the further use of T cells thus obtained for reintroduction into the patient.


SEQUENCE LISTING (8 sequences)

SEQ ID NO: 1 - protein, 48 aa, Artificial
  Derivative of the lacZ alpha peptide used as a flexible amino acid linker
MASSGGTGGSGGTGGSGGGGASPSLVPSSDPLVTAASVLEFALAGAQE

SEQ ID NO: 2 - protein, 26 aa, Artificial
  Synthetic flexible linker
GGGSGGGTGGSGGTGGSGGTGGSGGT

SEQ ID NO: 3 - protein, 25 aa, Artificial
  lacZ-based synthetic linker
EQAGALAFELVSAATVLPDSSPVLS

SEQ ID NO: 4 - protein, 8 aa, Artificial
  Synthetic linker
SGGSSGGG

SEQ ID NO: 5 - protein, 15 aa, Artificial
  Long synthetic linker
SSSGGGSSGGSSGGG

SEQ ID NO: 6 - protein, 8 aa, Artificial
  Octapeptide used to stabilize tetramerization
LLILAILH

SEQ ID NO: 7 - protein, 31 aa, Artificial
  Sequence of last 12 amino acids of MHC joined to first 12 amino acids of DsRed by a 7 amino acid linker
KLPPSTVSNTASGGSSGGGVASSENVITEFM

SEQ ID NO: 8 - protein, 40 aa, Artificial
  Sequence of last 12 amino acids of MHC joined to first 12 amino acids of DsRed with a linker encoding an epitope tag
KLPPSTVSNTASQPELAPEDPEDGGHDKVRSSKNVIKEFM